(19) United States
(12) Patent Application Publication
(10) Pub. No.: US 2010/0255065 A1
(43) Pub. Date: Oct. 7, 2010

(54) METHOD FOR DIFFERENCIATING MESENCHYMAL STEM CELL AND CULTURING CHONDROCYTES USING ALGINATE COATED FIBRIN/HA COMPOSITE SCAFFOLD

(75) Inventors: Byoung-Hyun Min, Seoul (KR); So Ra Park, Seoul (KR); Sang-Hyug Park, Gyeonggi-do (KR)

Correspondence Address: LUCAS & MERCANTI, LLP, 475 PARK AVENUE SOUTH, 15TH FLOOR, NEW YORK, NY, 10016, US

(73) Assignee: REGENPRIME CO., LTD., Suwon-si, Gyeonggi-do (KR)

(21) Appl. No.: 12/671,264

(22) PCT Filed: Aug. 1, 2007
(86) PCT No.: PCT/KR07/03710

§ 371 Date: Mar. 15, 2010

(51) Int. Cl. A61K 35/32; A61K 9/70; A61P 19/02; A61P 19/04; C12N 5/07; C12N 5/0775

(52) U.S. Cl. 424/443; 424/93.7; 435/402; 435/377

(57) ABSTRACT

The present invention relates to a method for differentiating mesenchymal stem cells and culturing chondrocytes using a fibrin/HA(hyaluronate) composite whose biocompatibility and durability are enhanced, and a therapeutic composition containing the fibrin/HA composite, and more particularly, to a method for culturing chondrocytes and differentiating mesenchymal stem cells into chondrocytes using an alginate-coated fibrin/HA composite gel and a composition for treating a cartilage disease and a composition for treating a disc disease using a fibrin/HA composite scaffold containing a fibrin degradation inhibitor. According to the present invention, disadvantages of the traditional fibrin/HA composite being reduced in size and easily degraded in a short time period during culture were overcome, so that cells can be cultured in a more stable environment. The treatment composition according to the present invention has superior biocompatibility and biodegradability and thus it can be used for effective treatment for cartilage diseases, and it can regenerate the nuclei pulposi of a new intervertebral disk unlike currently used surgical treatment of degenerative intervertebral disk disease, so that it is expected that fundamental treatment of disc diseases can be achieved.

### TECHNICAL FIELD

The present invention relates to a method for differentiating mesenchymal stem cells and culturing chondrocytes using a fibrin/HA(hyaluronate) composite whose biocompatibility and durability are enhanced, and a therapeutic composition containing the fibrin/HA composite, and more particularly, to a method for culturing chondrocytes and differentiating mesenchymal stem cells into chondrocytes using an alginate-coated fibrin/HA composite gel and a composition for treating a cartilage disease and a composition for treating a disc disease using a fibrin/HA composite scaffold containing a fibrin degradation inhibitor.

### BACKGROUND ART

To construct artificial cartilages using tissue engineering or cell therapy, studies on a method of using chondrocytes and a method of using mesenchymal stem cells are being conducted. Many methods for preparing cartilage tissue using chondrocytes, based on which in vivo experiments had been performed, are being reported, but a method for inducing chondrogenic differentiation of mesenchymal stem cells has not yet been clearly suggested. In order to construct tissue engineered cartilage using stem cells, scaffolds, cells and growth factors, which are the 3 essential factors in tissue engineering, should be appropriately applied to establish the conditions thereof. Scaffolds are generally divided into synthetic polymer scaffolds and natural polymer scaffolds. Since synthetic polymers are generally not preferable compared to natural polymers in terms of biocompatibility and biodegradability, many experiments using a natural polymer have been carried out.

Fibrin is a biodegradable natural polymer obtained by mixing thrombin to fibrinogen and solidifying the mixture, and is used for various purposes since it has been reported to show superior biocompatibility, biodegradability and conjugating ability to subchondral bones as a scaffold material. Fibrin of these properties has advantages for cartilage regeneration in that it provides an effective environment for the secretion of extracellular matrix while maintaining chondrocyte phenotype so that it facilitates cartilage tissue formation and attachment of constructs to a defect site. However, it also has disadvantages in that it has to be more solid in physical strength to be used as an ideal scaffold, and it could not fully provide differentiation environment for cells because of the fast degradation rate.

HA(hyaluronate) is a biopolymer, a non-sulfate glycosaminoglycan consisted of repeating unit of glucuronic acid/N-acetyl glucosamine disaccharide. It has been reported that, when used as a scaffold, HA is effective for forming an extracellular matrix since it promotes migration and proliferation of chondrocytes, and functions to inhibit the activity of cytokine IL-1β inducing cartilage tissue degradation. However, HA also has a problem in that it has perfect biocompatibility in its natural state, but the biocompatibility thereof decreases because it undergoes chemical reactions when it is combined with cells and transplanted in vivo.

We expected that fibrin and HA can be an ideal natural scaffold when combined together in view of the fact that fibrin structure can be stabilized when HA is added to fibrin. In addition, fibrin and HA, whose concentrations increase in the event of tissue injury, are key factors that play a significant role in the recovery processes. Accordingly, the present inventors have prepared a composite scaffold of fibrin and HA, and used them for differentiating mesenchymal stem cells. As a result, the fibrin/HA composite scaffold enabled mesenchymal stem cells to differentiate into chondrocytes without adding growth factors(KR 684940). However, there is a disadvantage in that the size of said fibrin/HA composite scaffold in vivo decreases with the passage of time and thus differentiation into chondrocytes is inhibited.

Until now, as three dimensional cultural methods for differentiating mesenchymal stem cells into chondrocytes, pellet culture and alginate bead and alginate layer cultures have been mainly used. Pellet culture is generally known to be effective in maintaining chondrocyte phenotype, and it can provide an extracellular environment similar to that for early cartilage tissue formation by easily adhering cells through centrifugation to induce cell adhesion. However, sufficient conditions for differentiation of stem cells into chondrocytes cannot be provided only by cell adhesion using centrifugation and thus the differentiation into chondrocytes (chondrogenesis) is mainly induced by adding growth factors such as TGF-β.

In addition, methods of inducing differentiation into chondrocytes using alginate has been recognized as an effective method for transplantation because it provides a physiologically suitable environment for cells by encapsulating cells in alginate beads, and a mechanical strength at which it can be easily handled. However, when stem cell differentiation is induced in laboratories through alginate culture, differentiation into chondrocytes is not sufficiently achieved if growth factor is not added, and satisfactory results in in vivo experiments have not also been reported.

TGF-β(transforming growth factor) mainly used in three dimensional culture is known as a material which plays an important role in developing bones and cartilages in vivo. Many experimental results have been reported that it is effective for differentiating mesenchymal stem cells of mouse, human, and rabbit sources into chondrocytes.

In the treatment of diseases, injuries or malformations affecting spinal motion segments, and especially those affecting disc tissue, it has long been known to remove some or all of a degenerated, ruptured or otherwise failing disc. In cases involving intervertebral disc tissue that has been removed or is otherwise absent from a spinal motion segment, corrective measures are indicated to ensure the proper spacing of the vertebrae formerly separated by the removed disc tissue.

In some instances, the two adjacent vertebrae are fused together using transplanted bone tissue, an artificial fusion component, or other compositions or devices. Spinal fusion procedures, however, have raised concerns in the medical community that the bio-mechanical rigidity of intervertebral fusion may predispose neighboring spinal motion segments to rapid deterioration. More specifically unlike a natural intervertebral disc, spinal fusion prevents the fused vertebrae from pivoting and rotating with respect to one another. Such lack of mobility tends to increase stresses on adjacent spinal motion segments. Additionally, several conditions may develop within adjacent spinal motion segments, including disc degeneration, disc herniation, instability, spinal stenosis, spondylosys and facet joint arthritis.

Thus, there is an urgent need to develop a method for regenerating intervertebral disks by means of a biological process not a surgical operation method.

Accordingly, the present inventors have made extensive efforts to develop a fibrin/HA composite whose biocompatibility and durability are enhanced for three dimensional cultures of chondrocytes and mesenchymal stem cells. As a result, the present inventors have found that, when a fibrin/HA composite gel coated with alginate and a fibrin/HA composite added with scaffold degradation inhibitors (i.e., aprotinin, EACA or elastinal) are used as a tissue engineering scaffold or a cell delivery carrier, the size reduction or degradation, which is observed in the traditional fibrin/HA composite gels, did not occur, so that it is possible to provide a stable culture environment to chondrocytes and promote mesenchymal stem cell differentiation, thereby completing the present invention.

### SUMMARY OF THE INVENTION

Therefore, it is an object of the present invention to provide a method for culturing chondrocytes using a fibrin/HA gel composite scaffold whose biocompatibility and durability are enhanced.

It is another object of the present invention to provide a method for differentiating mesenchymal stem cells using an alginate-coated fibrin/HA composite scaffold whose biocompatibility and durability are enhanced.

It is still another object of the present invention to provide a composition for treating a cartilage disease, which comprises a fibrin/HA composite scaffold having mesenchymal stem cells or chondrocytes attached thereto, whose biocompatibility and durability are enhanced.

It is yet another object of the present invention to provide a composition for treating a disk disease, which comprises a fibrin/HA composite scaffold having mesenchymal stem cells or chondrocytes attached thereto, whose biocompatibility and durability are enhanced.

To achieve the above object, the present invention provides a method for culturing chondrocytes, the method comprising the steps of: (a) mixing primary cultured chondrocytes and a fibrinogen/HA solution consisting of 50˜95 wt % of fibrinogen and 5˜50 wt % of HA; (b) preparing a fibrin/HA composite scaffold having chondrocytes attached thereto by adding CaCl₂, factor VIII and thrombin to the mixture solution; (c) coating the fibrin/HA composite scaffold having chondrocytes attached thereto, with alginate; and (d) culturing the chondrocytes attached to the fibrin/HA composite scaffold coated with alginate

In addition, the present invention provides a method for differentiating mesenchymal stem cells into chondrocytes, the method comprising the steps of: (a) mixing primary cultured mesenchymal stem cells and a fibrinogen/HA solution consisting of 50˜95 wt % of fibrinogen and 5˜50 wt % of HA; (b) preparing a fibrin/HA composite scaffold having mesenchymal stem cells attached thereto by adding CaCl₂, factor VIII and thrombin to the mixture solution; (c) coating the fibrin/HA composite scaffold having mesenchymal stem cells attached thereto, with alginate; and (d) culturing the mesenchymal stem cells attached to the fibrin/HA composite scaffold coated with alginate.

Also, the present invention provides a composition for treating a cartilage disease, which comprises, as an active ingredient, a fibrin/HA composite scaffold having mesenchymal stem cells or chondrocytes attached thereto, prepared by a method comprising the steps of: (a) mixing primary cultured mesenchymal stem cells or chondrocytes and a fibrinogen/HA solution consisting of 50˜95 wt % of fibrinogen and 5˜50 wt % of HA; (b) preparing a fibrin/HA composite scaffold having mesenchymal stem cells or chondrocytes attached thereto by adding CaCl₂, factor VIII and thrombin to the mixture solution; and (c) coating the fibrin/HA composite scaffold having mesenchymal stem cells or chondrocytes attached thereto, with alginate.

Moreover, the present invention provides a composition for treating a disc disease, which comprises, as an active ingredient, a fibrin/HA composite scaffold having mesenchymal stem cells or chondrocytes attached thereto, prepared by a method comprising the steps of (a) mixing primary cultured mesenchymal stem cells or chondrocytes and a fibrinogen/HA solution consisting of 50˜95 wt % of fibrinogen and 5˜50 wt % of HA; (b) preparing a fibrin/HA composite scaffold by adding CaCl₂, factor VIII and thrombin to the mixture solution; and (c) coating the fibrin/HA composite scaffold having mesenchymal stem cells or chondrocytes attached thereto, with alginate.

In addition, the present invention provides a method for culturing chondrocytes, the method comprising the steps of: (a) mixing primary cultured chondrocytes and a fibrinogen/HA solution consisting of 50˜95 wt % of fibrinogen and 5˜50 wt % of HA; (b) preparing a fibrin/HA composite scaffold having chondrocytes attached thereto by adding CaCl₂, factor VIII and thrombin to the mixture solution, and then a fibrin degradation inhibitor thereto; and (c) culturing the chondrocytes attached to the fibrin/HA composite scaffold.

The present invention also provides a method for differentiating mesenchymal stem cells into chondrocytes, the method comprising the steps of: (a) mixing primary cultured mesenchymal stem cells and a fibrinogen/HA solution consisting of 50˜95 wt % of fibrinogen and 5˜50 wt % of HA; (b) preparing a fibrin/HA composite scaffold by adding CaCl₂, factor VIII and thrombin to the mixture solution, and then a fibrin degradation inhibitor thereto; and (c) culturing the mesenchymal stem cells attached to the fibrin/HA composite scaffold.

The present invention also provides a composition for treating a cartilage disease, which comprises, as an active ingredient, a fibrin/HA composite scaffold having mesenchymal stem cells or chondrocytes attached thereto, prepared by a method comprising the steps of: (a) mixing primary cultured mesenchymal stem cells or chondrocytes and a fibrinogen/HA solution consisting of 50˜95 wt % of fibrinogen and 5˜50 wt % of HA; and (b) preparing a fibrin/HA composite scaffold by adding CaCl₂, factor VIII and thrombin to the mixture solution, and then a fibrin degradation inhibitor thereto.

The present invention also provides a composition for treating a disc disease, which comprises, as an active ingredient, a fibrin/HA composite scaffold having mesenchymal stem cells or chondrocytes attached thereto, prepared by a method comprising the steps of: (a) mixing primary cultured containing mesenchymal stem cells or chondrocytes and a fibrinogen/HA solution consisting of 50˜95 wt % of fibrinogen and 5˜50 wt % of HA; and (b) preparing a fibrin/HA composite scaffold by adding CaCl₂, factor VIII and thrombin to the mixture solution, and then adding a fibrin degradation inhibitor thereto.

In addition, the present invention provides a method for culturing chondrocytes, the method comprising the steps of: (a) mixing primary cultured chondrocytes and a fibrinogen/HA solution consisting of 50˜95 wt % of fibrinogen and 5˜50 wt % of HA; (b) preparing a fibrin/HA composite scaffold by adding CaCl₂, factor VIII and thrombin to the mixture solution, and then a fibrin degradation inhibitor thereto; (c) coating the fibrin/HA composite scaffold having chondrocytes attached thereto, with alginate; and (d) culturing the chondrocytes attached to the fibrin/HA composite scaffold coated with alginate.

The present invention also provides a method for differentiating mesenchymal stem cells into chondrocytes, the method comprising the steps of: (a) mixing primary cultured mesenchymal stem cells and a fibrinogen/HA solution consisting of 50˜95 wt % of fibrinogen and 5˜50 wt % of HA; (b) preparing a fibrin/HA composite scaffold having mesenchymal stem cells attached thereto by adding CaCl₂, factor VIII and thrombin to the mixture solution, and then a fibrin degradation inhibitor thereto; (c) coating the fibrin/HA composite scaffold having mesenchymal stem cells attached thereto, with alginate; and (d) culturing the mesenchymal stem cells attached to the fibrin/HA composite scaffold, with the alginate.

Moreover, the present invention provides a composition for treating a cartilage disease, which comprises, as an active ingredient, a fibrin/HA composite scaffold having mesenchymal stem cells or chondrocytes attached thereto, prepared by a method comprising the steps of: (a) mixing primary cultured mesenchymal stem cells or chondrocytes and a fibrinogen/HA solution consisting of 50˜95 wt % of fibrinogen and 5˜50 wt % of HA; (b) preparing a fibrin/HA composite scaffold by adding CaCl₂, factor VIII and thrombin to the mixture solution, and then a fibrin degradation inhibitor thereto; and (c) coating the fibrin/HA composite scaffold having mesenchymal stem cells or chondrocytes attached thereto, with alginate.

Also, the present invention provides a composition for treating a disc disease, which comprises, as an active ingredient, a fibrin/HA composite scaffold having mesenchymal stem cells or chondrocytes attached thereto, prepared by a method comprising the steps of: (a) mixing primary cultured containing mesenchymal stem cells or chondrocytes and a fibrinogen/HA solution consisting of 50˜95 wt % of fibrinogen and 5˜50 wt % of HA; (b) preparing a fibrin/HA composite scaffold by adding CaCl₂, factor VIII and thrombin to the mixture solution, and then adding a fibrin degradation inhibitor thereto; and (c) coating the fibrin/HA composite scaffold having mesenchymal stem cells or chondrocytes attached thereto, with alginate.

The above and other objects, features and embodiments of the present invention will be more clearly understood from the following detailed description and accompanying claims.

### BRIEF DESCRIPTION OF THE DRAWINGS

FIG. 1 is photographs showing changes in appearance according to the culture period of the inventive fibrin/HA composite gel coated with alginate and a non-coated fibrin/HA composite gel.

FIG. 2 is a histological image analysis showing the results of Safranin-O staining (x200, x40) and alcian blue staining (x40) of the inventive fibrin/HA composite gel coated with alginate and a non-coated fibrin/HA composite gel in vitro and in vivo.

FIG. 3 is image analysis (x200) of the expression of Type II collagen and HIF-1α using immunohistochemistry in the inventive fibrin/HA composite gel coated with alginate and a non-coated fibrin/HA composite gel in vitro.

FIG. 4 shows the total GAG and hydroxyproline contents in the inventive fibrin/HA composite gel coated with alginate and a non-coated fibrin/HA composite gel in vitro and in vivo.

FIG. 5 shows glucose (a) and NO₂⁻ (b) concentrations in culture media of the inventive fibrin/HA composite gel coated with alginate and a non-coated fibrin/HA composite gel in vitro.

FIG. 6 shows the whole shape (a), the results of protein secretion (b) and fibrinolytic zymography (c) in a fibrin/HA composite gel group without adding cells in vitro.

FIG. 7 is the measurement results showing the mechanical intensity of a fibrin/HA composite gel coated with alginate and a non-coated fibrin/HA composite gel in in vivo transplantation.

FIG. 8 shows images and changes in volume of a fibrin/HA composite gel added with a fibrin degradation inhibitor, which is cultured in vitro.

FIG. 9 shows images and changes in volume of a fibrin/HA composite gel added with a fibrin degradation inhibitor, which is transplanted in vivo.

FIG. 10 shows histological and biochemical analysis results of a fibrin/HA composite gel added with a fibrin degradation inhibitor.

FIG. 11 shows mechanical intensity analysis results of a fibrin/HA composite gel added with a fibrin degradation inhibitor.

FIG. 12 is photographs showing the degree of regeneration of the nuclei pulposi of intervertebral disks into which the fibrin/HA composite gel according to the present invention is transplanted, which is observed by naked eyes.

FIG. 13 is the histological measurement results showing the degree of regeneration of the nuclei pulposi of intervertebral disks into which the fibrin/HA composite gel according to the present invention is transplanted, which were obtained by Safranin O/Fast green staining.

FIG. 14 shows the degree of regeneration of the nuclei pulposi of intervertebral disks into which the fibrin/HA composite gel according to the present invention is transplanted, which is observed using a simple X-ray examination.

### DETAILED DESCRIPTION OF THE INVENTION, AND PREFERRED EMBODIMENTS

In one aspect, the present invention relates to a method for culturing chondrocytes, the method comprising the steps of: (a) mixing primary cultured chondrocytes and a fibrinogen/HA solution consisting of 50˜95 wt % of fibrinogen and 5˜50 wt % of HA; (b) preparing a fibrin/HA composite scaffold having chondrocytes attached thereto by adding CaCl₂, factor VIII and thrombin to the mixture solution; (c) coating the fibrin/HA composite scaffold having chondrocytes attached thereto, with alginate; and (d) culturing the chondrocytes attached to the fibrin/HA composite scaffold coated with alginate.

In another aspect, the present invention relates to a method for differentiating mesenchymal stem cells into chondrocytes, the method comprising the steps of: (a) mixing primary cultured mesenchymal stem cells and a fibrinogen/HA solution consisting of 50˜95 wt % of fibrinogen and 5˜50 wt % of HA; (b) preparing a fibrin/HA composite scaffold having mesenchymal stem cells attached thereto by adding CaCl₂, factor VIII and thrombin to the mixture solution; (c) coating the fibrin/HA composite scaffold having mesenchymal stem cells attached thereto, with alginate; and (d) culturing the mesenchymal stem cells attached to the fibrin/HA composite scaffold coated with alginate.

It has been confirmed that the HA used in the present invention serves as an essential element in normal cartilage matrix, and increases the number of chondrocytes and matrix synthesis, and the higher the molecular weight of HA is, the more effect on chondrocyte differentiation the HA has (Goodstone, N. J. et al., Tissue Eng., 10:621, 2004).

Therefore, in the present invention, the molecular weight of the HA is preferably 500˜10,000 kD.

In an embodiment of the present invention, the coating in the step (c) is preferably carried out by soaking the fibrin/HA composite scaffold into a sodium alginate solution, and the coating is repeated 2˜10 times, thus preparing a fibrin/HA composite scaffold having a suitable thickness of alginate coating layer formed thereon to use.

In the inventive method for differentiating mesenchymal stem cells, mesenchymal stem cells can be differentiated into chondrocytes with high efficiency even without adding growth factors such as TGF-β, etc., but it is also allowed to add growth factors and in the case of adding growth factors, it is preferable to add TGF-β1, IGF-1, BMP-2 and ascorbic acid.

In the present invention, the mesenchymal stem cells are preferably derived from embryos, adult tissue or born marrow.

In still another aspect, the present invention relates to a composition for treating a cartilage disease, which comprises, as an active ingredient, a fibrin/HA composite scaffold having mesenchymal stem cells or chondrocytes attached thereto, prepared by a method comprising the steps of: (a) mixing primary cultured mesenchymal stem cells or chondrocytes and a fibrinogen/HA solution consisting of 50˜95 wt % of fibrinogen and 5˜50 wt % of HA; (b) preparing a fibrin/HA composite scaffold having mesenchymal stem cells or chondrocytes attached thereto by adding CaCl₂, factor VIII and thrombin to the mixture solution; and (c) coating the fibrin/HA composite scaffold having mesenchymal stem cells or chondrocytes attached thereto, with alginate.

In the present invention, the cartilage disease is preferably selected from the group consisting of degenerative joint disease, rheumatoid arthritis, and fracture.

In further another aspect, the present invention relates to a composition for treating a disc disease, which comprises, as an active ingredient, a fibrin/HA composite scaffold having mesenchymal stem cells or chondrocytes attached thereto, prepared by a method comprising the steps of: (a) mixing primary cultured mesenchymal stem cells or chondrocytes and a fibrinogen/HA solution consisting of 50˜95 wt % of fibrinogen and 5˜50 wt % of HA; (b) preparing a fibrin/HA composite scaffold by adding CaCl₂, factor VIII and thrombin to the mixture solution; and (c) coating the fibrin/HA composite scaffold having mesenchymal stem cells or chondrocytes attached thereto, with alginate.

The composition for treating a disk disease in the present invention is an alternative agent for repair of damaged nuclei pulposi in vertebrate animals, which can be inserted using a surgical operation.

In an embodiment of the present invention, the coating in the step (c) is preferably carried out by soaking the fibrin/HA composite scaffold into a sodium alginate solution, and the coating is repeated 2-10 times, thus preparing a fibrin/HA composite scaffold having a suitable thickness of alginate coating layer formed thereon.

The inventive fibrin/HA composite scaffold having alginate coating layer formed thereon has advantages in that it provides a matrix having excellent compatibility to chondrocytes and mesenchymal stem cells in vivo, and it is a natural polymer and thus it can be biodegradable.

Alginate coating didn't show changes in size in a fibrin/HA composite gel containing no cells (in serum-free media and 10% FBS DMEM), a fibrin/HA composite gel coated with alginate showed higher strength than that of a non-coated fibrin/HA composite gel.

In addition, it was observed that protein secretion in a non-coated fibrin/HA composite gel increased compared with a fibrin/HA composite gel coated with alginate, suggesting that alginate coating inhibits the degradation of fibrin/HA composite gel in vivo.

As result of histological, immunohistochemical and chemical analyses, chondrofication in a fibrin/HA composite gel coated with alginate was actively progressing compared with a non-coated fibrin/HA composite gel. Particularly, Type II collagen showed an apparent tendency for increased expression in groups with 2- and 4-layer coatings.

It was also found that the alginate coated group has the highest mechanical intensity among the groups with the same transplantation periods.

More glucose is consumed in a medium in which the inventive fibrin/HA composite gel coated with alginate is cultured, which is because oxygen consumption within articular cartilage is inhibited by glucose and, when oxygen concentration is inhibited glucose will be consumed by chondrocytes. Thus, an increase in glucose consumption indicates that chondrocytes are in an active state.

It was observed that the synthesis of NO in the inventive fibrin/HA composite gel group coated with alginate is extremely inhibited. Since NO synthesis inhibitors inhibit the degradation of extracellular matrix in cartilage, NO synthesis inhibition is an important factor in maintaining extracellular matrix (ECM). Therefore, NO synthesis inhibition by the fibrin/HA composite gel coated with alginate indicates that said fibrin/HA composite gel coated with alginate provides a suitable environment for cartilage formation by chondrocytes.

In another aspect, the present invention relates to a method for culturing chondrocytes, the method comprising the steps of: (a) mixing primary cultured chondrocytes and a fibrinogen/HA solution consisting of 50˜95 wt % of fibrinogen and 5˜50 wt % of HA; (b) preparing a fibrin/HA composite scaffold having chondrocytes attached thereto by adding CaCl₂, factor VIII and thrombin to the mixture solution, and then a fibrin degradation inhibitor thereto; and (c) culturing the chondrocytes attached to the fibrin/HA composite scaffold.

In still another aspect, the present invention relates to a method for differentiating mesenchymal stem cells into chondrocytes, the method comprising the steps of: (a) mixing primary cultured mesenchymal stem cells and a fibrinogen/HA solution consisting of 50˜95 wt % of fibrinogen and 5˜50 wt % of HA; (b) preparing a fibrin/HA composite scaffold by adding CaCl₂, factor VIII and thrombin to the mixture solution, and then a fibrin degradation inhibitor thereto; and (c) culturing the mesenchymal stem cells attached to the fibrin/HA composite scaffold.

In the present invention, the fibrin degradation inhibitor is preferably elastatinal and the fibrin degradation inhibitor preferably additionally comprises EACA (epsilon aminocaproic acid) or aprotinin.

In an embodiment of the present invention, it was confirmed that when said fibrin degradation inhibitor is added to the fibrin/HA composite, a size decrease of a transplant with the culture time could be prevented, physical strength thereof was improved, and it was more effectively differentiated into chondrocytes.

In the present invention, said EACA is preferably added at a concentration of 0.1˜10 mg/ml, aprotinin is preferably added at a concentration of 1˜100 μl/ml, and elastatinal is preferably added at a concentration of 1 μl/ml˜1 mg/ml. If it is added at a lower concentration than the above mentioned concentration, the ability to inhibit fibrin degradation will be lost, and if it is added at a higher concentration than the above mentioned concentration, there will be a possibility of showing cellular toxicity.

In the present invention, the step (b) is preferably performed by additionally adding a growth factor selected from the group consisting of TGF-β, IGF-1, BMP-2 and ascorbic acid.

In another aspect, the present invention relates to a composition for treating a cartilage disease, which comprises, as an active ingredient, a fibrin/HA composite scaffold having mesenchymal stem cells or chondrocytes attached thereto, prepared by a method comprising the steps of: (a) mixing primary cultured mesenchymal stem cells or chondrocytes and a fibrinogen/HA solution consisting of 50˜95 wt % of fibrinogen and 5˜50 wt % of HA; and (b) preparing a fibrin/HA composite scaffold by adding CaCl₂, factor VIII and thrombin to the mixture solution, and then a fibrin degradation inhibitor thereto.

In still another aspect, the present invention relates to a composition for treating a disc disease, which comprises, as an active ingredient, a fibrin/HA composite scaffold having mesenchymal stem cells or chondrocytes attached thereto, prepared by a method comprising the steps of: (a) mixing primary cultured containing mesenchymal stem cells or chondrocytes and a fibrinogen/HA solution consisting of 50˜95 wt % of fibrinogen and 5˜50 wt % of HA; and (b) preparing a fibrin/HA composite scaffold by adding CaCl₂, factor VIII and thrombin to the mixture solution, and then adding a fibrin degradation inhibitor thereto.

In yet another aspect, the present invention relates to a method for culturing chondrocytes, the method comprising the steps of: (a) mixing primary cultured chondrocytes and a fibrinogen/HA solution consisting of 50˜95 wt % of fibrinogen and 5˜50 wt % of HA; (b) preparing a fibrin/HA composite scaffold by adding CaCl₂, factor VIII and thrombin to the mixture solution, and then a fibrin degradation inhibitor thereto; (c) coating the fibrin/HA composite scaffold having chondrocytes attached thereto, with alginate; and (d) culturing the chondrocytes attached to the fibrin/HA composite scaffold coated with alginate.

In further another aspect, the present invention relates to a method for differentiating mesenchymal stem cells into chondrocytes, the method comprising the steps of: (a) mixing primary cultured mesenchymal stem cells and a fibrinogen/HA solution consisting of 50˜95 wt % of fibrinogen and 5˜50 wt % of HA; (b) preparing a fibrin/HA composite scaffold having mesenchymal stem cells attached thereto by adding CaCl₂, factor VIII and thrombin to the mixture solution, and then a fibrin degradation inhibitor thereto; (c) coating the fibrin/HA composite scaffold having mesenchymal stem cells attached thereto, with alginate; and (d) culturing the mesenchymal stem cells attached to the fibrin/HA composite scaffold, with the alginate.

In still further aspect, the present invention relates to a composition for treating a cartilage disease, which comprises, as an active ingredient, a fibrin/HA composite scaffold having mesenchymal stem cells or chondrocytes attached thereto, prepared by a method comprising the steps of: (a) mixing primary cultured mesenchymal stem cells or chondrocytes and a fibrinogen/HA solution consisting of 50˜95 wt % of fibrinogen and 5˜50 wt % of HA; (b) preparing a fibrin/HA composite scaffold having mesenchymal stem cells or chondrocytes attached thereto by adding CaCl₂, factor VIII and thrombin to the mixture solution, and then a fibrin degradation inhibitor thereto; and (c) coating the fibrin/HA composite scaffold having mesenchymal stem cells or chondrocytes attached thereto with alginate.

In yet another further aspect, the present invention relates to a composition for treating a disc disease, which comprises, as an active ingredient, a fibrin/HA composite scaffold, prepared by a method comprising the steps of: (a) mixing primary cultured mesenchymal stem cell or chondrocytes and a fibrinogen/HA solution consisting of 50˜95 wt % of fibrinogen and 5˜50 wt % of HA; (b) preparing a fibrin/HA composite scaffold by adding CaCl₂, factor VIII and thrombin to the mixture solution, and then a fibrin degradation inhibitor thereto; and (c) coating the fibrin/HA composite scaffold having mesenchymal stem cells or chondrocytes attached thereto, with alginate.

### EXAMPLES

The present invention will hereinafter be described in further detail by examples. However, it is to be understood that these examples can be modified into other various forms, and the scope of the present invention is not intended to be limited to such examples.

In particular, although the following examples only describe a method using a fibrin/HA composite as a method for disc regeneration according to the present invention, it is obvious to a person skilled in the art that disk regeneration surgery can be carried out using a fibrin/HA composite coated with alginate.

### Example 1

### Isolation of Chondrocytes

Chondrocytes were isolated from rabbit articular cartilage. Mail rabbits weighing 250 g were put into euthanasia by an overdose of Nembutal injection, and the knee joints were sterilely dissected. The knee joints were finely diced to wash with PBS (phosphate buffered saline), and then treated with 0.2% collagenase (Worthington Biochemical, USA) dissolved in a PBS solution for 5 hours at 37° C. The solutions containing the post-treated cells were filtered through a 70 μm nylon filter (Falcon, USA) and then the filtrate was centrifuged at 1200 rpm for ten minutes to obtain cell pellets. The obtained pellet was washed with PBS 2 times, and suspended in DMEM(Dulbecco's modified eagles medium, Gibco BRL, USA) containing 10% FBS(fetal bovine serum, Gibco BRL, USA), 100 U/ml penicillin G (Gibco BRL, USA) and 100 μg/ml streptomycin (Gibco BRL, USA).

Both cell number and viabilities were determined by trypan blue assay and the cells were dispersed in plain medium at a density of 1.5×10⁵ cells/cm², then, cultured in a 5% CO₂ incubator at 37° C. The culture medium was replaced everyday and primary chondrocytes were subcultured twice to use in the experiments.

### Example 2

### Preparation of Alginate-Coated Fibrin/Ha Composite Gel

To prepare a fibrin/HA composite gel, the cells prepared in Example 1 were centrifuged to form pellets, then suspended in a solution containing 9˜18 mg/ml fibrinogen (GreenCross, Korea) and 10 mg/ml of 3000 kDa HA (hyaluronate; LGCI, Korea). 5×10⁶ cells/ml of chondrocyte suspension was homogenized with a solution containing aprotinin (GreenCross, Korea), 60 U/ml thrombin (1000 U/mg: Sigma, USA), a fibrin stabilizing factor XIII (GreenCross, Korea) and 50 Mm CaCl₂.

250 μl of the fibrin/HA composite solution was dropped into a Petri dish to form a gel, and, for alginate coating, the gel pieces were soaked in 1 ml of sterile 100 mM CaCl₂ solution for 10 minutes to immerse in a sodium alginate solution (Sigma, 2.4% in 0.15M NaCl), thus forming a solid alginate coating layer having a thickness of 0.5 mm. The coated fibrin/HA composite gel was washed with 0.15M NaCl, and finally rinsed with CDM (chondrogenic defined medium, Table. 1). Each gel was transferred to a 6-well culture plate and cultured in CDM medium (in vitro culture experiments).

Experimental groups were divided into 4 groups [group 1: a non-coated fibrin/HA composite gel (F/H), group 2: a gel coated with a single layer of alginate (F/H+1AC), group 3: a gel coated with 2-layer alginate coatings (F/H+2AC), and group 4: a gel coated with 4-layer alginate coatings (F/H+4AC)].

TABLE 1
Composition of CDM Medium

| Component | Concentration | |
|---|---|---|
| high glucose DMEM (Gibco BRL, USA) | base | |
| antibiotic-antimycotic (Gibco BRL, USA) | 1% | |
| insulin | 1.0 | mg/mL |
| selenious acid | 0.5 | mg/mL |
| transferin | 0.55 | mg/mL |
| ascorbic acid | 50 | μg/mL |
| dexamethasone | 100 | mM |
| proline | 40 | μg/mL |
| BSA(bovine serum albumin) | 1.25 | mg/mL |
| sodium pyruvate | 100 | μg/mL |

### Example 3

### Transplantation into Nude Mice

An alginate coated fibrin/HA composite gel and a non-coated fibrin/HA composite gel were soaked in a culture medium for 2 hours before being transplanted into mice. The fibrin/HA composite gels of the 2 groups were subcutaneously injected at the same time into the back of 15 nude mice per each group under sterile conditions. Five mice were sacrificed at the 1st, 2nd and 4th week after transplantation, respectively, and water content was measured by weighing the extracted fibrin/HA composite gel.

### Example 4

### Characteristic Analysis for Fibrin/HA Composite Gel

### 4-1. Size Changes

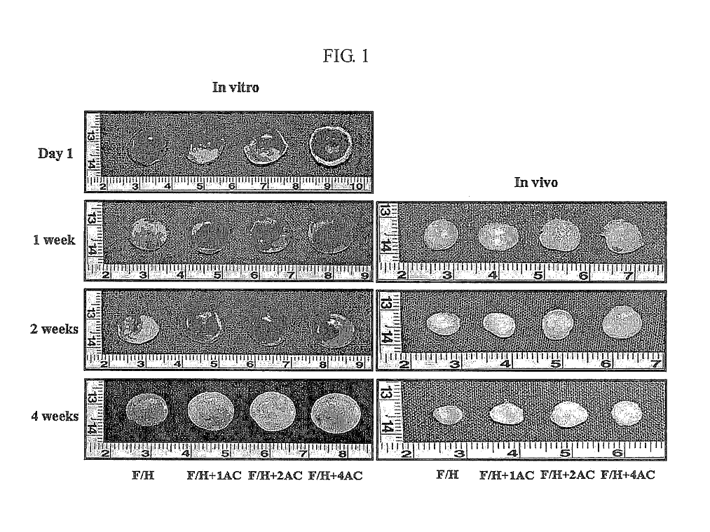

While any significant differences in sizes were not observed in vitro in the alginate coated fibrin/HA composite gel groups, the size of the non-coated fibrin/HA composite gel group significantly reduced by 35% at the first week compared with its original size. In vivo transplantation experiment, any significant differences were not observed in the coated group and the non-coated group until the 1st week, and the size differences were shown with the passage of time, especially, the fibrin/HA composite gels having 2- and 4-layer coatings were 1.5-fold larger than others (FIG. 1).

### 4-2. Histological and Immunohistochemical Analysis

After observing with an optical microscope, tissues were fixed in a 4% formalin fixation solution for 24 hours. Alginate was removed from the alginate coated tissue samples using 2% sodium citrate and the alginate coated tissue samples were fixed. Then, samples were embedded in paraffin and sectioned into a thickness of 4 μm. The serial sections were stained with Safranin-O and alcian blue to confirm proteoglycan sulfation.

In the whole groups, cells which were contained in the fibrin/HA composite gels were uniformly distributed in the cellular matrix. As a result of observing through Safranin-O staining, cartilage-specific lacunae were widely formed throughout the composite gel.

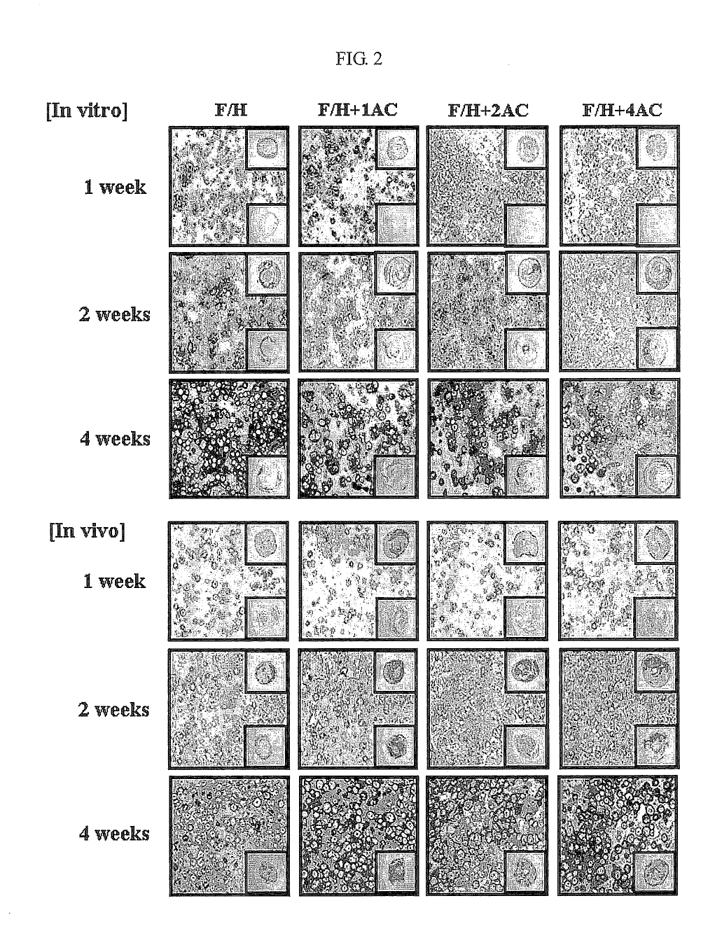

Also, from the result of alcian blue strain, sulfated proteoglycans accumulated in the same group were observed. Any negative influences were not observed in tissues of alginate-coated fibrin/HA composite gel groups, which have been cultured in vitro (FIG. 2).

In vivo transplantation, the fibrin/HA composite gels in all groups showed positive staining for cartilage-specific ECM. As a result of Safranin-O stain, while the week proteoglycan accumulation was observed in the peripheral region of the F/H group, positive staining showing a dark color was observed in the same region of the alginate coated group. The chondrocytes of fibrin/HA composite gel were not affected by alginate coating. The cells were nicely forming cartilage (FIG. 2).

The expression of Type II collagen and HIF-1α as a major ECM protein and a hypoxia marker were examined by imminohistochemical analysis. The sections were subsequently washed with 70% ethanol and PBS, then treated with 3% H₂O₂/PBS to add 0.15% Triton X-100. After the resulting sections were blocked with 1% BSA, they were allowed to react with mouse anti-human type II collagen (1:500, Chemicon, USA) for one hour, and then allowed to additionally react with biotnylated secondary antibody. Finally, proteins were detected using horseradish peroxidase-conjugated avidin system (Vector Laboratories, USA). The immunostained sections were counter-stained with Mayer's hematoxylin (Sigma, USA) to observe with a microscope (Nikon E600, Japan).

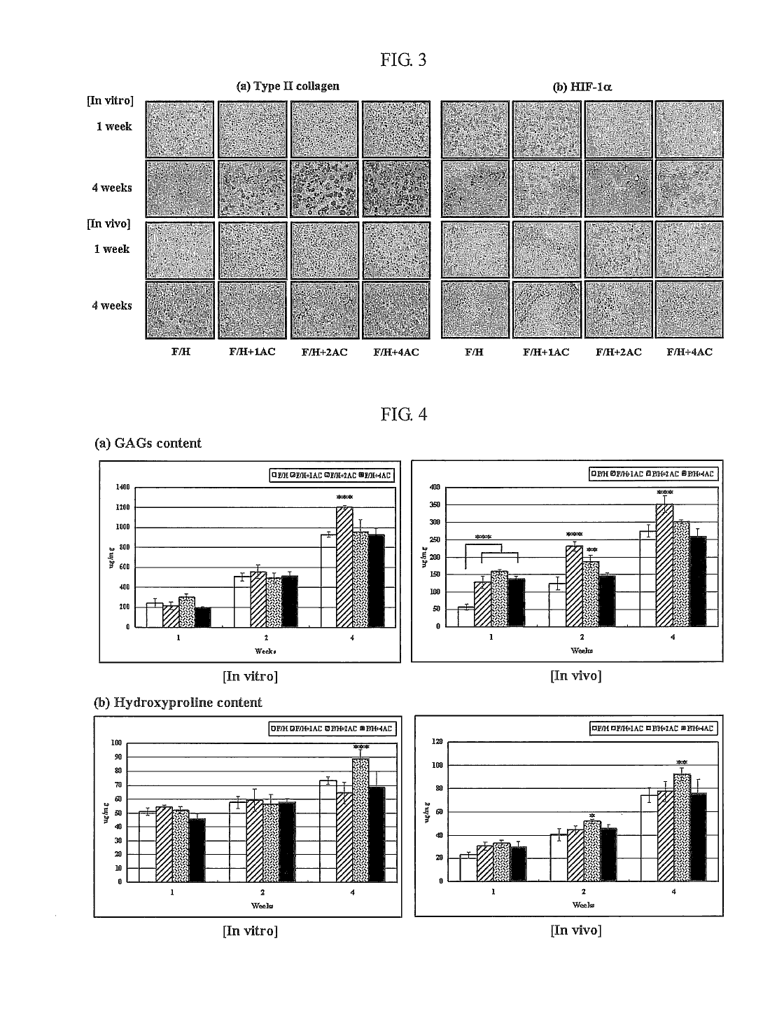

Type II collagen was observed in samples of all groups at the 4^{th} week, and showed an apparent tendency for increased expression in fibrin/HA composite gel groups having 2- and 4-layer coatings, which have been cultured in vitro (FIG. 3a). Meanwile, the expression of HIF-1α didn't show any differences in all groups regardless of in vitro and in vivo conditions (FIG. 3b).

### 4-3. Analysis of GAG and Hydroxyproline Contents

Samples were degraded in papain solution (125 μg/ml papain, 5 mM L-cystein, 100 mM Na₂HPO₄, 5 mM EDTA, pH 6.2) at a temperature of 60° C. for 16 hours. The total GAG concentration was analyzed with DMB (1,9-dimethylmethylene blue) assay. Each sample was mixed with the DMB solution to measure an absorbance at 225 nm. The total GAG in each sample was estimated by a standard curve using 0˜5 μg/ml shark condroitin sulfate (Sigma, USA).

The hydroxyproline content was measured by Stegemann-Stalder's method (Stegemann, H. and Stalder, K., Clin. Chim. Acta, 18:267, 1967).

First, a lyophilized sample was homogenized in distilled water (added with papain protease) using a homogenizer to prepare a standard hydroxyproline (0˜50 μg) solution. The sample was mixed with sodium hydroxide (final concentration 2N) to be total of 50 μl to hydrolyze at 120° C. for 20 minutes, and added with 450 μl chloramines-T, thus oxidizing it at room temperature for 25 minutes. Then, 500 μl Enrlich's aldehyde reagent was added to each sample and allowed to react at 65° C. for 20 minutes, thus developing chromophores. Finally, absorbance was measured at 550 nm using a ELISA READER.

Data were expressed as average ±tandard deviation per each hour. Comparison of a control group and an experimental group was made by one-way analysis (ANOVA) using GraphPad Instat program (GraphPad software Inc, USA).

The total GAG and hydroxypoline contents were increased with the passage of time during in vitro and in vivo culture. In in vitro culture, the average ECM concentration was 927.8±27.5 (μg/mg dry weight) GAG in F/H group at 4^{th} week, and 1202.47±17.65 (μg/mg dry weight) GAG in F/H+1AC group (FIG. 4a).

Hydroxyproline content was 73.2±2.7 (μg/mg dry weight) in F/H group at 4^{th} week, and 89.1±5.95 (μg/mg dry weight) in F/H+2AC group (FIG. 4b). From the above results, it was observed that F/H+2AC group showed a statistically significant difference compared with other groups, and showed a similar pattern to in vitro culture upon in vivo transplantation.

In particular, more positive effects on GAG synthesis were shown in the alginate coated groups compared to the F/H group at the 1^{st} week. Hydroxyproline content measured in vivo showed the highest level (92.53±5.6) in F/H+2AC group. It was defined that p<0.001 was statistically significant for GAG and p<0.01 was statistically significant for hydroxyproline

### 4-4. Analysis of Culture Medium

Glucose and nitrite contents used for analysis of chondrocyte metabolism were measured by collecting exchange media from each gel, and the concentration of fibrin-degrading enzyme was measured using ELISA READER (BIO-TEK, Instruments, INC., USA).

The glucose content was measured using glucose assay reagent (Sigma, USA) comprising hexokinase, glucose-6-phosphate dehydrogenase and NAD+.240 μl of the reagent was added in 12 μl of each medium sample or a standard reagent (0˜1.2 g/L glucose) and allowed to react at 37° C. for 3 minutes to measure absorbance at 340 nm.

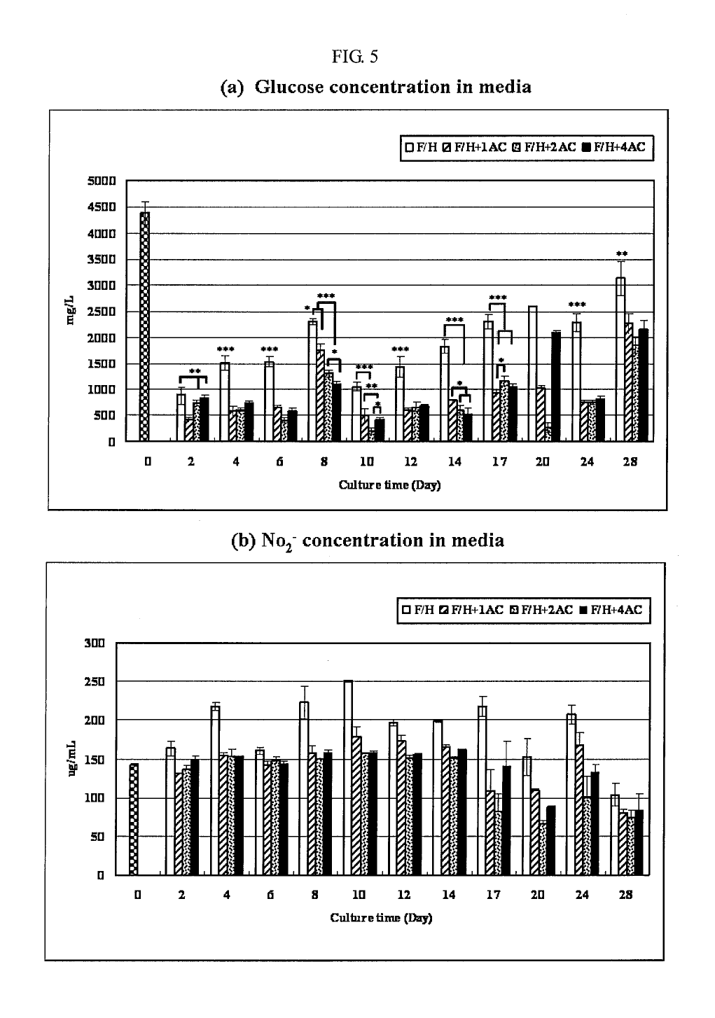

As a result, it was observed that the glucose concentration in the non-coated gel group was higher than that in alginate coated fibrin/HA composite gel group (FIG. 5a).

The nitrite (NO₂—) is produced as nitric acid is degraded, and the content thereof was measured using the Griess reagent system (Invitrogen, USA). First, 50 μl of sample medium was dispersed onto each well to add 50 μl of sulfanilamide solution, and left to stand at room temperature for 10 minutes to add NED solution, then left to stand in a dark place for 10 minutes. Finally, absorbance was measured within 30 minutes at 520˜550 nm. The nitrite concentration of the sample was estimated with a standard curve measured using nitrite standard solution (0˜100 μM).

As a result, the nitrite level in the medium was significantly low in the alginate coated group compared with the non-coated group. Especially, the nitrogen production level in F/H+2AC was the lowest among all groups (FIG. 5b).

### 4-5. Observation of Gel Size for Degradation Velocity Measurement and Protein Secretion Analysis

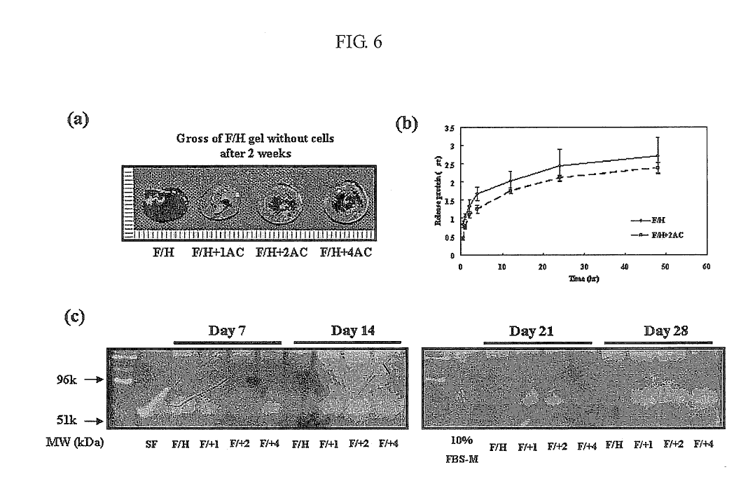

When all groups were cultured in a serum-free media for 2 weeks, specific changes in sizes of the gels were not observed. However, the gels of F/H+2AC group and the F/H+4AC group were harder and stronger (FIG. 6a).

The amount of protein secreted was measured using ELISA (enzyme linked immunosorbent assay) for 48 hours. First, a diluted albumin (BSA) standard solution was prepared such that it is similar to sample buffer. 100 μl of each standard solution and culture medium were transferred into a 96 well plate, and 100 μl of working reagent was added to each well to mix with a plate shaker for 30 seconds, and allowed to react at 37° C. for 30 minutes. The absorbance of the sample was measured at 562 nm.

The amount of protein secreted from the F/H group and the F/H+2AC group showed a significant difference depending on the culture time, and the F/H group secreted more protein (FIG. 6b).

### 4-6. Fibrinolytic Zymography

Fibrinolytic zymography in a medium was measured in the presence of active degrading enzyme. The medium sample was electrophoresed at 10 mA using 125 SDS-PAGE gel copolymerized by adding 0.12% fibrin having a thickness of 0.75 mm. Gel after electrophoresis was washed with 50 mM Tris buffer (pH 7.4) containing 2.5% Triton X to remove SDS, then washed with distilled water for 30 minutes. After washing, the gel was allowed to react with 30 mM Tris buffer (pH 7.4) containing 200 mM NaCl, 10 mM CaCl₂ and 0.02% NaN₃ at 37° C. for 16 hours. The gel after the reaction was immobilized with 30% methanol for 1 hour, and then fibrin gel was stained with Coomassie blue stain. Protein migration was confirmed by comparing with a low molecular range marker.

As a result, the activity of fibrin degrading enzymes showed no differences in all groups, and the degrading enzyme activities of the alginate coated group and the non-coated group showed similar patterns.

### 4.7 Analysis of Mechanical Intensity

For samples collected from in vivo transplantation experiment, unconfined compression test was performed using Universal Testing Machine (Model H5K-T, H.T.E., England). Each sample placed on the bottom platen of the compression tester was compressed at a rate of 1 mm/min, and moved along the length programmed between the top platen and the bottom platen, and then stopped automatically. Once the peak load was obtained from a load-displacement curve, statistical analysis was performed for each compressive power.

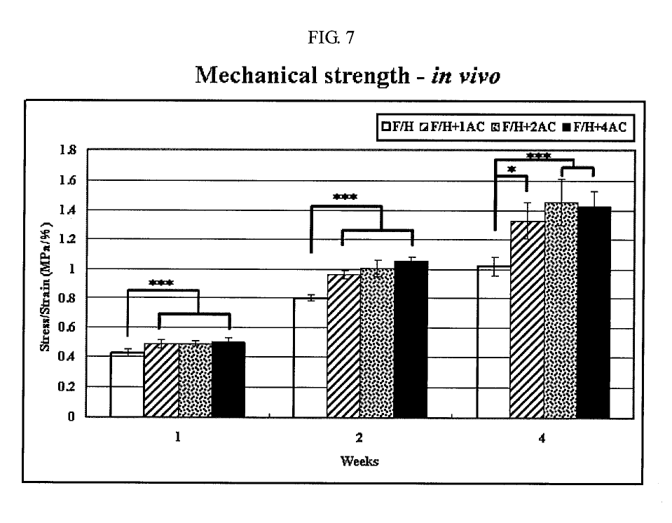

As a result, it was seen that the alginate coated group was stronger than the non-coated group, the differences in the compressive power among the alginate coated groups were not found (FIG. 7).

### Example 5

### Preparation of Fibrin/Ha Composite Gel Added with Fibrin Degradation Inhibitor

The cells prepared in Example 1 were centrifuged to form pellets, then suspended in a solution containing 9˜18 mg/ml of fibrinogen (GreenCross, Korea) and 10 mg/ml of 3000 kDa HA (hyaluronate; LGCI, Korea). 5×10⁶ cells/ml of chondrocyte suspension was homogenized with a solution comprising aprotinin (GreenCross, Korea), 60 U/ml thrombin (1000 U/mg: Sigma, USA), a fibrin stabilizing factor XIII (GreenCross, Korea) and 50 mM CaCl₂.

250 μl of the fibrin/HA composite solution was dropped into a petri dish to form a gel, then added with fibrin degradation inhibitor, EACA (epsilon aminocaproic acid), aprotinin and elastatinal by dividing them into experimental groups shown in Table 2.

TABLE 2
Composition of experimental groups added with fibrin degradation
inhibitor

| | F | F/A | F/D | F/E | F/A + D | F/A + E | F/D + E | F/A + D + E |
|---|---|---|---|---|---|---|---|---|
| Aprotinin | X | ◯ | X | X | ◯ | ◯ | X | ◯ |
| (20 μg/ml) | | | | | | | | |
| Elastinal | X | X | ◯ | X | ◯ | X | ◯ | ◯ |
| (50 μg/ml) | | | | | | | | |
| EACA (2 mg/ml) | X | X | X | ◯ | X | ◯ | ◯ | ◯ |

The fibrin/HA composite gels in the each experimental group were cultured in a 12 well plate containing DMEM (10% NCS) for 2 weeks, thus measuring changes in the size and property thereof.

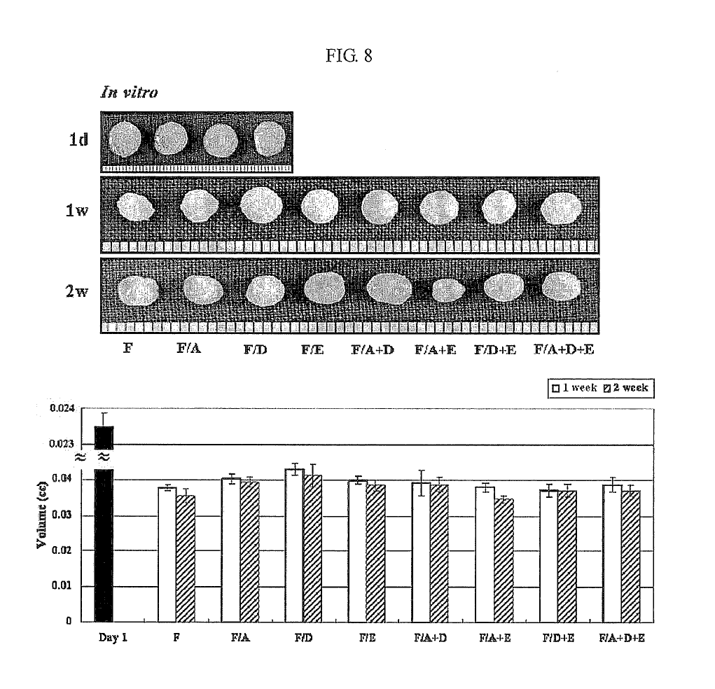

As a result, the size of the fibrin/HA composite gel as the control group was reduced compared to the other fibrin/HA composite gel groups added with fibrin degradation inhibitors, and the size reduction was observed at the 1^{st} week of culture. At the 2^{nd} week, the group added with aprotinin and EACA showed the smallest size, and all experimental groups appeared more shiny than the groups at the 1^{st} week (FIG. 8).

### Example 6

### Transplantation of Fibrin/HA Composite Gel Added with Fibrin Degradation Inhibitor into Nude Mice

The fibrin/HA composite gels added with fibrin degradation inhibitor, which is prepared in Example 5, were subcutaneously transplanted into the back of 18 nude mice. The transplanted mice were sacrificed at the 1^{st}, 2^{nd}, and 4^{th} week to collect transplants.

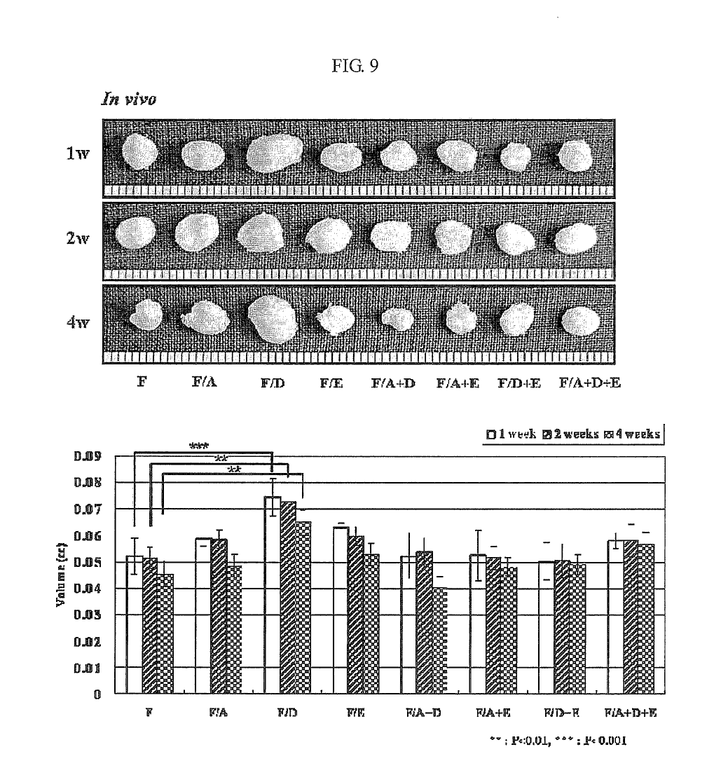

As a result of measuring the volume of the transplants, the group added with elastinal (F/D) showed the least size reduction, and this tendency was notably confirmed at the 4^{th} week of transplantation. However, the size of the F/A+D group was extremely reduced in spite of elastinal addition (FIG. 9).

### Example 7

### Property Analysis of Fibrin/HA Composite Gel Added with Fibrin Degradation Inhibitor

For a fibrin/HA composite gel added with fibrin degradation inhibitor, histological and biochemical analysis was performed and the strength thereof was measured using the same method as described Example 4.

As a result, at the 2^{nd} week of transplantation, the F/D and F/E groups showed excellent results compared with the other groups. All experimental groups formed a structure similar to that of natural cartilage, and showed positive staining in Safranin-O staining and Alcian Blue staining during the whole period. It was confirmed that GAG accumulation increased with the passage of time.

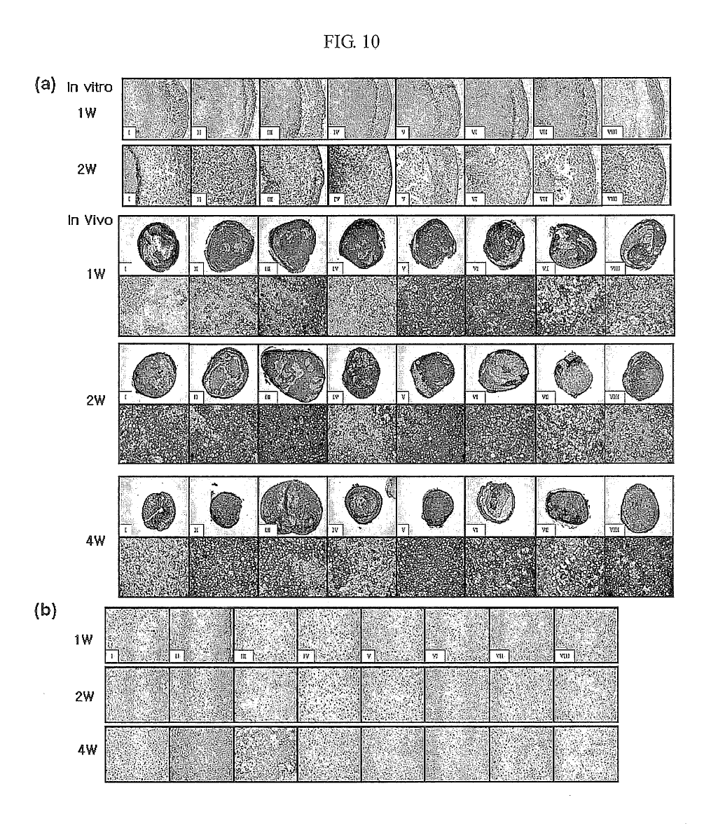

Differentiated chondrocytes within lacunae and cartilage formation was observed in more than 90% of the area at the 4^{th} week. In Alcian Blue staining, strong positive staining was observed. (FIG. 10a)

Although more Type II collagen was expressed in the F/A and F/D groups (FIG. 10 b), most significantly, volume changes, metachromatic staining of the affected cartilage and Type II collagen expression were observed in the F/D group.

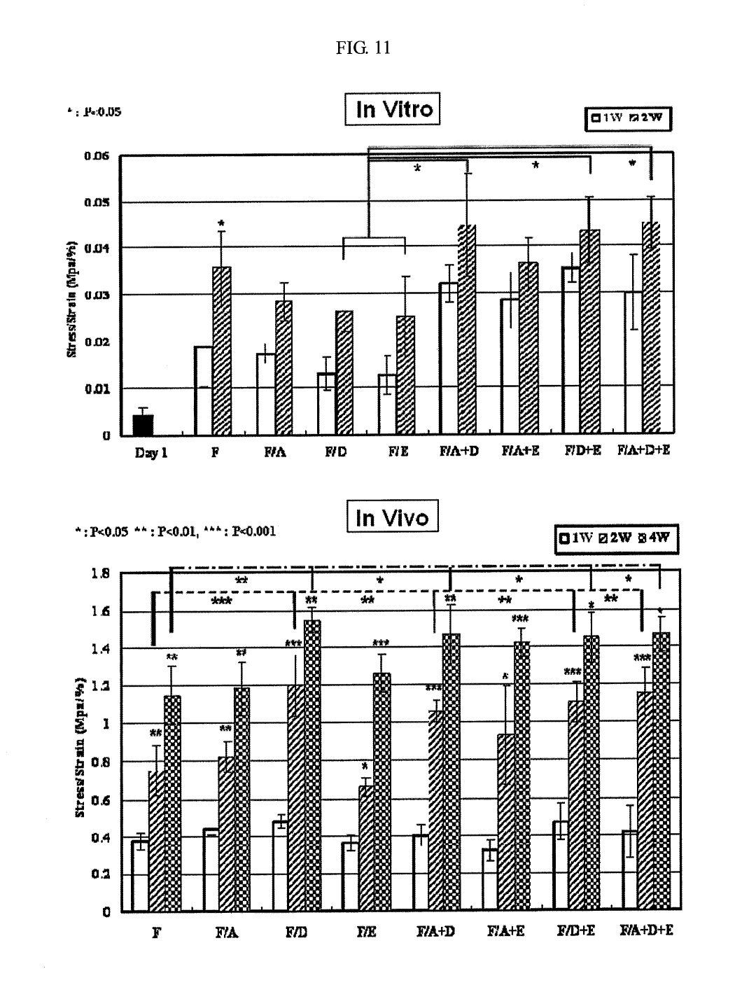

As a result of examining mechanical intensity, in the fibrin/HA composite gels cultured for 1 week and 2 weeks in vitro, groups added with a fibrin the degradation inhibitor (F/A, F/D, F/E) showed no significant difference in compressive power, but groups added with a combination of more than two fibrin degradation inhibitors (F/A+D, F/A+E, F/D+E, F/A+D+E) showed strong rigidity. Also, the mechanical intensity increased during the period of transplantation into nude mice. Especially, groups added with ealastinal (F/D, F/A+D, F/D+E, F/A+D+E) showed very strong rigidity compared with the control group (FIG. 11).

### Example 8

### Construction of an Animal Model of Disc Degeneration and Transplantation of a Fibrin/HA Composite Scaffold

SD white mice (300 g) were anesthetized with ketamine, and the tail was sterilized with betadin solution, followed by incising the skin overlying the intervertebral disks to expose the intervertebral disks. The nuclei pulposi of the intervertebral disc was removed using 22G injection needle, and 20 μl of fibrin/HA composite gel (F/A+D+) containing fibrin degradation inhibitor, which is prepared in Example 5, was injected into the nuclei pulposi, then the skin was sutured with 4-0 nylon thread, thus breeding the mice for 8 weeks.

Experimental groups were divided into 3 groups, herein the group 1 was a control group in which the nuclei pulposi was removed therefrom and other treatments were not applied thereto, the group 2 was a group in which the nuclei pulposi was removed therefrom and only the fibrin/HA composite was transplanted thereinto, and the group 3 was a group in which the nuclei pulposi was removed therefrom and the fibrin/HA+cell composite was transplanted thereinto. After transplantation, the mice were sacrificed at 2nd, 4th, and 8th week and analysis was performed.

### Example 9

### Naked Eye Examination and Histological Examination

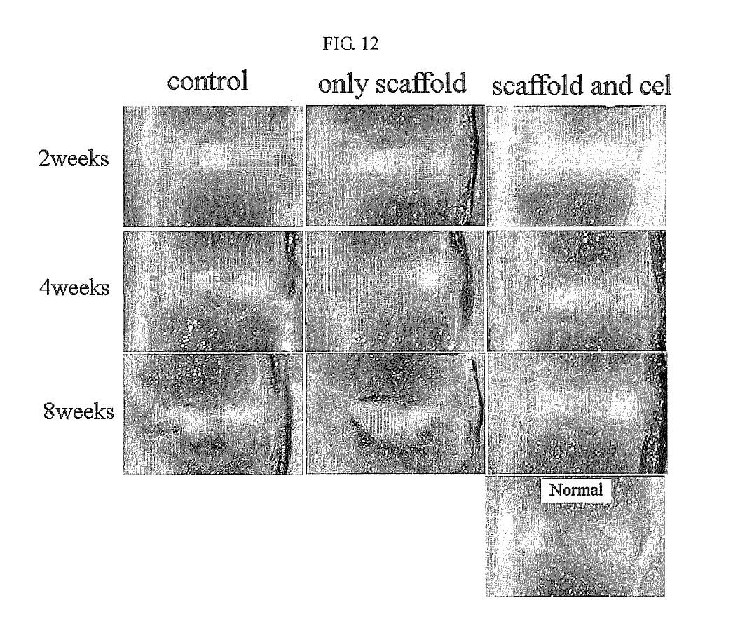

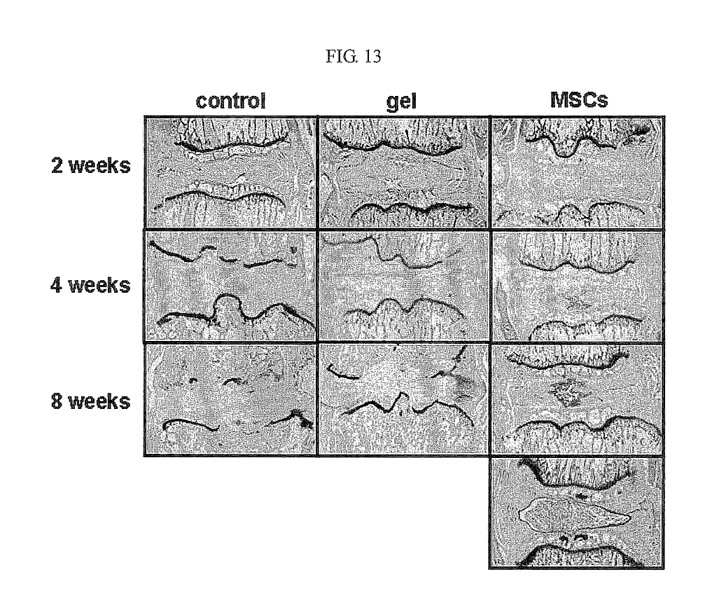

It was observed that, in the group into which fibrin/HA+cell composite is transplanted, the nuclei pulposi of an interverbral disk regenerated gradually, and especially, at 8^{th} week, a significant regeneration effect was seen compared to the control group and the group into which only the fibrin/HA composite was transplanted (FIG. 12).

The intervertebral disk tissues obtained at the 2nd, 4th, and 8th week after transplantation were cut along the cross section and the longitudinal section, and subjected to Safranin O/Fast green staining, thus observing formation of protein polysaccharides.

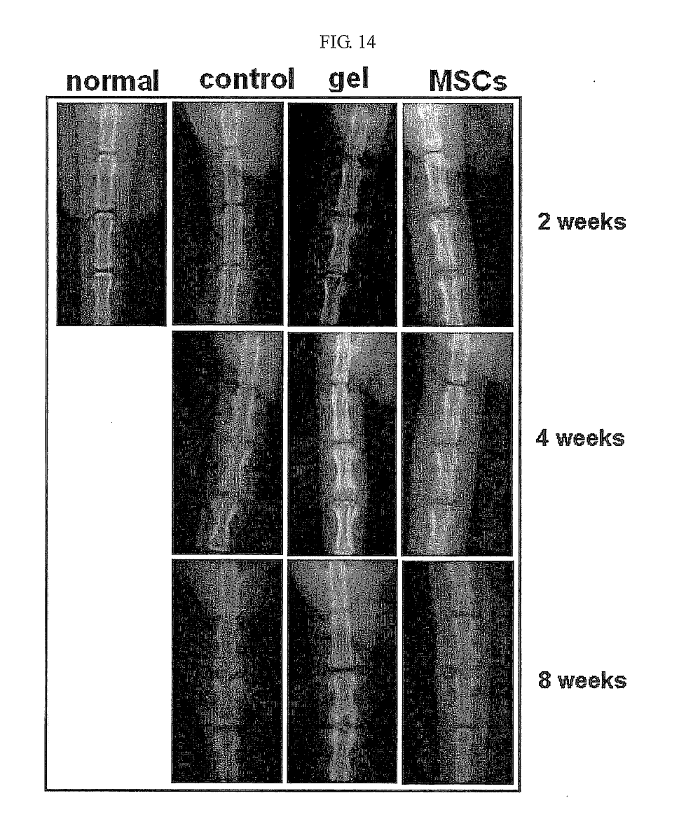

As a results of observing changes in the intervertebral disc height through a simple X-ray examination, osteophyte formation around the intervertebral disk, and the like, it was observed that, in the group into which the fibrin/HA and stem cells were transplantted, the intervertebral disk height was well maintained (FIG. 14). However, any significant difference between the control group and the group into which only the fibrin/HA was transplanted was not shown.

In addition, the results of the present example provide an experimental basis for pre-clinical testing to apply the fibrin/HA+stem cell or chondrocyte composite to clinical practice for treating intervertebral disk diseases, and it is expected that a novel method for biological cell treatment, which is basically different from currently used surgical treatment of degenerative intervertebral disk disease, can be developed.

### INDUSTRIAL APPLICABILITY

As described above, the present invention has the effects to provide a method for culturing chondrocytes and differentiating mesenchymal stem cells into chondrocytes using a fibrin/HA composite scaffold whose biocompatibility and durability are enhanced, and the effect to provide a composition for treating a cartilage disease and a composition for treating a disc, which comprise a fibrin/HA composite scaffold having mesenchymal stem cells or chondrocytes attached thereto, whose biocompatibility and durability are enhanced.

According to the present invention, disadvantages of the traditional fibrin/HA composite being reduced in size and easily degraded in a short time period during culture were overcome, so that cells can be cultured in a more stable environment. The treatment composition according to the present invention has superior biocompatibility and biodegradability and thus it can be used for effective treatment for cartilage diseases, and it can regenerate the nuclei pulposi of a new intervertebral disk unlike currently used surgical treatment of degenerative intervertebral disk disease, so that it is expected that fundamental treatment of disc diseases can be achieved.

While the present invention has been described with reference to the particular illustrative embodiment, it is not to be restricted by the embodiment but only by the appended claims. It is to be appreciated that those skilled in the art can change or modify the embodiment without departing from the scope and spirit of the present invention.

What is claimed is:

1. A method for culturing chondrocytes, the method comprising the steps of:
(a) mixing primary cultured chondrocytes and a fibrinogen/HA solution consisting of 50˜95 wt % of fibrinogen and 5˜50 wt % of HA;
(b) preparing a fibrin/HA composite scaffold having chondrocytes attached thereto by adding CaCl₂, factor VIII and thrombin to the mixture solution;
(c) coating the fibrin/HA composite scaffold having chondrocytes attached thereto, with alginate; and
(d) culturing the chondrocytes attached to the fibrin/HA composite scaffold coated with alginate.

2. The method according to claim 1, wherein the molecular weight of the HA is 500˜10,000 kD.

3. The method according to claim 1, wherein the coating in the step (c) is carried out by soaking the fibrin/HA composite scaffold having the chondrocytes attached thereto into a sodium alginate solution, and the coating is repeated 2-10 times.

4. The method according to claim 1, wherein the step (b) or (d) is carried out by additionally adding a growth factor selected from the group consisting of TGF-β1, IGF-1, BMP-2 and ascorbic acid.

5. A method for differentiating mesenchymal stem cell into chondrocytes, the method comprising the steps of:
(a) mixing primary cultured mesenchymal stem cells and a fibrinogen/HA solution consisting of 50˜95 wt % of fibrinogen and 5˜50 wt % of HA;
(b) preparing a fibrin/HA composite scaffold having mesenchymal stem cells attached thereto by adding CaCl₂, factor VIII and thrombin to the mixture solution;
(c) coating the fibrin/HA composite scaffold having mesenchymal stem cells attached thereto, with alginate; and
(d) culturing the mesenchymal stem cells attached to the fibrin/HA composite scaffold coated with alginate.

6. The method according to claim 5, wherein the molecular weight of the HA is 500˜10,000 kD.

7. The method according to claim 5, wherein the mesenchymal stem cells are derived from embryos, adult tissue or born marrow.

8. The method according to claim 5, wherein the coating in the step (c) is carried out by soaking the fibrin/HA composite scaffold having the mesenchymal stem cells attached thereto into a sodium alginate solution, and the coating is repeated 2˜10 times.

9. The method according to claim 5, wherein the step (b) or (d) is carried out by additionally adding a growth factor selected from the group consisting of TGF-β, IGF-1, BMP-2 and ascorbic acid.

10. A composition for treating a cartilage disease, which comprises, as an active ingredient, a fibrin/HA composite scaffold having mesenchymal stem cells or chondrocytes attached thereto, prepared by a method comprising the steps of: (a) mixing primary cultured mesenchymal stem cells or chondrocytes and a fibrinogen/HA solution consisting of 50˜95 wt % of fibrinogen and 5˜50 wt % of HA; (b) preparing a fibrin/HA composite scaffold having mesenchymal stem cells or chondrocytes attached thereto by adding CaCl₂, factor VIII and thrombin to the mixture solution; and (c) coating the fibrin/HA composite scaffold having mesenchymal stem cells or chondrocytes attached thereto, with alginate.

11. The method according to claim 10, wherein the coating in the step (c) is carried out by soaking the fibrin/HA composite scaffold having mesenchymal stem cells or chondrocytes attached thereto into a sodium alginate solution, and the coating is repeated 2˜10 times.

12. The method according to claim 10, wherein the cartilage disease is selected from the group consisting of degenerative joint disease, rheumatoid arthritis, and fracture.

13. The method according to claim 10, wherein the step (b) or (d) is carried out by additionally adding a growth factor selected from the group consisting of TGF-β1, IGF-1, BMP-2 and ascorbic acid.

14. A composition for treating a disc disease, which comprises, as an active ingredient, a fibrin/HA composite scaffold having mesenchymal stem cells or chondrocytes attached thereto, prepared by a method comprising the steps of: (a) mixing primary cultured mesenchymal stem cells or chondrocytes and a fibrinogen/HA solution consisting of 50˜95 wt % of fibrinogen and 5˜50 wt % of HA; (b) preparing a fibrin/HA composite scaffold by adding CaCl₂, factor VIII and thrombin to the mixture solution; and (c) coating the fibrin/HA composite scaffold having mesenchymal stem cells or chondrocytes attached thereto, with alginate.

15. A method for culturing chondrocytes, the method comprising the steps of:
(a) mixing primary cultured chondrocytes and a fibrinogen/HA solution consisting of 50˜95 wt % of fibrinogen and 5˜50 wt % of HA;
(b) preparing a fibrin/HA composite scaffold having chondrocytes attached thereto by adding CaCl₂, factor VIII and thrombin to the mixture solution, and then a fibrin degradation inhibitor thereto; and
(c) culturing the chondrocytes attached to the fibrin/HA composite scaffold.

16. The method according to claim 15, wherein the molecular weight of the HA is 500˜10,000 kD.

17. The method according to claim 15, wherein the fibrin degradation inhibitor is elastatinal.

18. The method according to claim 17, wherein the fibrin degradation inhibitor additionally comprises EACA (epsilon aminocaproic acid) or aprotinin.

19. The method according to claim 15, wherein the step (b) or (c) is carried out by additionally adding a growth factor selected from the group consisting of TGF-β1, IGF-1, BMP-2 and ascorbic acid.

20. A method for culturing mesenchymal stem cell, the method comprising the steps of:
(a) mixing primary cultured mesenchymal stem cells and a fibrinogen/HA solution consisting of 50˜95 wt % of fibrinogen and 5˜50 wt % of HA;
(b) preparing a fibrin/HA composite scaffold by adding CaCl₂, factor VIII and thrombin to the mixture solution, and then a fibrin degradation inhibitor thereto; and
(c) culturing the mesenchymal stem cells attached to the fibrin/HA composite scaffold having mesenchymal stem cells attached thereto.

21. The method according to claim 20, wherein the fibrin degradation inhibitor is elastatinal.

22. The method according to claim 21, wherein the fibrin degradation inhibitor additionally comprises EACA (epsilon aminocaproic acid) or aprotinin.

23. The method according to claim 20, wherein the step (b) or (c) is carried out by additionally adding a growth factor selected from the group consisting of TGF-β1, IGF-1, BMP-2 and ascorbic acid.

24. The method according to claim 20, wherein the molecular weight of the HA is 500˜10,000 kD.

25. The method according to claim 20, wherein the mesenchymal stem cells are derived from embryos, adult tissue or born marrow.

26. A composition for treating a cartilage disease, which comprises, as an active ingredient, a fibrin/HA composite scaffold having mesenchymal stem cells or chondrocytes attached thereto, prepared by a method comprising the steps of: (a) mixing primary cultured mesenchymal stem cells or chondrocytes and a fibrinogen/HA solution consisting of 50˜95 wt % of fibrinogen and 5˜50 wt % of HA; and (b) preparing a fibrin/HA composite scaffold by adding CaCl₂, factor VIII and thrombin to the mixture solution, and then a fibrin degradation inhibitor thereto.

27. The composition according to claim 26, wherein the fibrin degradation inhibitor is elastatinal.

28. The composition according to claim 27, wherein the fibrin degradation inhibitor additionally comprises EACA (epsilon aminocaproic acid) or aprotinin.

29. The composition according to claim 26, wherein the step (b) is carried out by additionally adding a growth factor selected from the group consisting of TGF-β1, IGF-1, BMP-2 and ascorbic acid.

30. The composition according to claim 26, wherein the cartilage disease is selected from the group consisting of degenerative joint disease, rheumatoid arthritis, and fracture.

31. A composition for treating a disc disease, which comprises, as an active ingredient, a fibrin/HA composite scaffold having mesenchymal stem cells or chondrocytes attached thereto, prepared by a method comprising the steps of: (a) mixing primary cultured containing mesenchymal stem cells or chondrocytes and a fibrinogen/HA solution consisting of 50˜95 wt % of fibrinogen and 5˜50 wt % of HA; and (b) preparing a fibrin/HA composite scaffold by adding CaCl₂, factor VIII and thrombin to the mixture solution, and then adding a fibrin degradation inhibitor thereto.

32. The composition according to claim 31, wherein the fibrin degradation inhibitor is elastatinal.

33. The composition according to claim 32, wherein the fibrin degradation inhibitor additionally comprises EACA (epsilon aminocaproic acid) or aprotinin.

34. The composition according to claim 31, wherein the step (b) is carried out by additionally adding a growth factor selected from the group consisting of TGF-β1, IGF-1, BMP-2 and ascorbic acid.

35. The composition according to claim 31, wherein the cartilage disease is selected from the group consisting of degenerative joint disease, rheumatoid arthritis, and fracture.

36. A method for culturing chondrocytes, the method comprising the steps of:
(a) mixing primary cultured chondrocytes and a fibrinogen/HA solution consisting of 50˜95 wt % of fibrinogen and 5˜50 wt % of HA;
(b) preparing a fibrin/HA composite scaffold by adding CaCl₂, factor VIII and thrombin to the mixture solution, and then a fibrin degradation inhibitor thereto;
(c) coating the fibrin/HA composite scaffold having chondrocytes attached thereto, with alginate; and
(d) culturing the chondrocytes attached to the fibrin/HA composite scaffold coated with alginate.

37. The method according to claim 36, wherein the molecular weight of the HA is 500˜10,000 kD.

38. The method according to claim 36, wherein the fibrin degradation inhibitor is elastatinal.

39. The method according to claim 38, wherein the fibrin degradation inhibitor additionally comprises EACA (epsilon aminocaproic acid) or aprotinin.

40. The method according to claim 36, wherein the step (b) or (d) is carried out by additionally adding a growth factor selected from the group consisting of TGF-β1, IGF-1, BMP-2 and ascorbic acid.

41. A method for differentiating mesenchymal stem cells into chondrocytes, the method comprising the steps of:
(a) mixing primary cultured mesenchymal stem cells and a fibrinogen/HA solution consisting of 50˜95 wt % of fibrinogen and 5˜50 wt % of HA;
(b) preparing a fibrin/HA composite scaffold having mesenchymal stem cells attached thereto by adding CaCl₂, factor VIII and thrombin to the mixture solution, and then a fibrin degradation inhibitor thereto;
(c) coating the fibrin/HA composite scaffold having mesenchymal stem cells attached thereto, with alginate; and
(d) culturing the mesenchymal stem cells attached to the fibrin/HA composite scaffold coated with the alginate.

42. The method according to claim 41, wherein the fibrin degradation inhibitor is elastatinal.

43. The method according to claim 42, wherein the fibrin degradation inhibitor additionally comprises EACA (epsilon aminocaproic acid) or aprotinin.

44. The method according to claim 41, wherein the step (b) or (d) is carried out by additionally adding a growth factor selected from the group consisting of TGF-β1, IGF-1, BMP-2 and ascorbic acid.

45. The method according to claim 41, wherein the molecular weight of the HA is 500˜10,000 kD.

46. The method according to claim 5, wherein the mesenchymal stem cells are derived from embryos, adult tissue or born marrow.

47. A composition for treating a cartilage disease, which comprises, as an active ingredient, a fibrin/HA composite scaffold having mesenchymal stem cells or chondrocytes attached thereto, prepared by a method comprising the steps of: (a) mixing primary cultured mesenchymal stem cells or chondrocytes and a fibrinogen/HA solution consisting of 50˜95 wt % of fibrinogen and 5˜50 wt % of HA; (b) preparing a fibrin/HA composite scaffold having mesenchymal stem cells or chondrocytes attached thereto by adding CaCl₂, factor VIII and thrombin to the mixture solution, and then a fibrin degradation inhibitor thereto; and (c) coating the fibrin/HA composite scaffold having mesenchymal stem cells or chondrocytes attached thereto, with alginate.

48. The composition according to claim 47, wherein the fibrin degradation inhibitor is elastatinal.

49. The composition according to claim 48, wherein the fibrin degradation inhibitor additionally comprises EACA (epsilon aminocaproic acid) or aprotinin.

50. The composition according to claim 47, wherein the step (b) or (d) is carried out by additionally adding a growth factor selected from the group consisting of TGF-β1, IGF-1, BMP-2 and ascorbic acid.

51. The composition according to claim 47, wherein the cartilage disease is selected from the group consisting of degenerative joint disease, rheumatoid arthritis, and fracture.

52. A composition for treating a disc disease, which comprises, as an active ingredient, a fibrin/HA composite scaffold, prepared by a method comprising the steps of: (a) mixing primary cultured mesenchymal stem cell or chondrocytes and a fibrinogen/HA solution consisting of 50˜95 wt % of fibrinogen and 5˜50 wt % of HA; (b) preparing a fibrin/HA composite scaffold by adding CaCl₂, factor VIII and thrombin to the mixture solution, and then a fibrin degradation inhibitor thereto; and (c) coating the fibrin/HA composite scaffold having mesenchymal stem cells or chondrocytes attached thereto, with alginate.

53. The composition according to claim 52, wherein the fibrin degradation inhibitor is elastatinal.

54. The composition according to claim 53, wherein the fibrin degradation inhibitor additionally comprises EACA (epsilon aminocaproic acid) or aprotinin.

55. The composition according to claim 52, wherein the step (b) or (d) is carried out by additionally adding a growth factor selected from the group consisting of TGF-β1, IGF-1, BMP-2 and ascorbic acid.

56. The composition according to claim 47, wherein the cartilage disease is selected from the group consisting of degenerative joint disease, rheumatoid arthritis, and fracture.
